(19) United States
(12) Patent Application Publication
(10) Pub. No.: US 2026/0278797 A1
(43) Pub. Date: Sep. 17, 2026

(54) Method Of Obtaining Lengths From Images Representing A Section Of A Tissue Volume

(71) Applicant: COMPREMIUM AG, Bern (CH)

(72) Inventors: Peter Nuot Frei, Dulliken (CH); Vincent Boris Baumann, Gümligen (CH); Patrick Roth, Burgdorf (CH); Ulrich Baumann, Münsingen (CH); Federico Milani, Bern (CH)

(73) Assignee: COMPREMIUM AG, Bern (CH)

(21) Appl. No.: 19/168,530

(22) PCT Filed: Mar. 27, 2024
(86) PCT No.: PCT/EP2024/058391

§ 371 Date: Sep. 24, 2025

(30) Foreign Application Priority Data
Mar. 31, 2023 (CH) CH000357/2023

(51) Int. Cl. G06T 7/00; A61B 8/00; G06T 7/60

(52) U.S. Cl. CPC G06T 7/0016; G06T 7/60; A61B 8/4254; G06T 2207/10132; G06T 2207/30004

(57) ABSTRACT

For obtaining a first length from a first two-dimensional image representing a three-dimensional tissue volume section at a first time, and a second length from a second two-dimensional image representing a three-dimensional tissue volume section at a second time, first and second points are marked in the first image. Fourth and fifth points are marked in the second image. Whether a position and orientation of the first and the second images sufficiently match is checked by using the marked first, second, fourth and fifth points, and third and sixth data obtained from the first and second images, respectively. If so, the first and second lengths are determined from the first and second marked points and the third data, and the fourth and fifth marked points and the sixth data, respectively. The third and sixth points may correspond to physiological landmarks visible in the first and second images, respectively.

### TECHNICAL FIELD

The invention relates to a method of obtaining a first length from a first two-dimensional image representing a section of a three-dimensional tissue volume at a first time, and a second length from a second two-dimensional image representing a section of a three-dimensional tissue volume at a second time.

### BACKGROUND ART

There are various applications where the properties or the development of physiological conditions are examined or monitored based on data obtained from medical imaging. The images may be obtained e.g. by ultrasound, magnetic resonance, x-ray or OCT imaging. In many cases, the size of structures recognizable in the images will be determined and form an important basis for the further assessment of the physiological condition. Usually, the size will be characterized by one or several lengths.

A method including the measurement of a first length at a first point in time and the measurement of a second length at a second point in time is described in co-pending PCT/EP2022/072593 of the same applicant. It is used for the examination of the temporal progression of the elasticity of a tissue structure.

Often, corresponding results obtained from two or more images taken at different points in time and/or at different external or internal influences such as different applied external pressure, different phase of blood circulation or breathing cycle, etc., shall be compared. Usually, such a comparison is meaningful only if the tissue volume represented by the first image and by the second image and the measuring modalities (e.g. the orientation an axis along which a length value is determined) sufficiently match.

### SUMMARY OF THE INVENTION

It is the object of the invention to create a method pertaining to the technical field initially mentioned, that allows for a reliable comparison of length values obtained from two or more two-dimensional images representing a section of a three-dimensional tissue volume.

The solution of the invention is specified by the features of claim 1. According to the invention, the method comprises the steps of:
a) marking a first point in the first two-dimensional image;
b) marking a second point in the first two-dimensional image;
c) obtaining third data relating to the first two-dimensional image;
d) marking a fourth point in the second two-dimensional image;
e) marking a fifth point in the second two-dimensional image;
f) obtaining sixth data relating to the second two-dimensional image;
g) checking, using the marked first, second, fourth and fifth points and the third and sixth data, whether a position and orientation of the first two-dimensional image and the second two-dimensional image sufficiently match; and
if yes
(h) determining the first length from the first and second marked point and the third data; and
i) determining the second length from the fourth and fifth marked point and the sixth data.

The first, second, fourth and fifth points may be marked manually, by a user, using a suitable user interface. Marking the points may be effected by directly marking point-like locations (such as pixels in image data) in the first or second two-dimensional image, respectively. The marking step may be facilitated by providing cursors, crosshairs or similar visual aids. All or some of the points may be indirectly marked by marking extended 1- and/or 2-dimensional structures such as:
lines or arrows, where a single point is mapped to a designated location on the line or arrow, e. g. the center point or a designated end point or where two points are mapped to two designated locations, in particular the two end points;
arcs, where a single point is mapped to a designated location on the arc, e. g. the center point on the arc, a rotational center of the arc or a designated end point or where two points are mapped to two designated locations, in particular the two end points of the arc;
shapes such as circles, triangles, rectangles, etc., where a single point is mapped to a designated location, e. g. the center point of a circle, a triangle or a rectangle or a designated corner of a polygon, where two points are mapped to the center and a further designated location or to two designated locations on the perimeter, or where three points are mapped to the center and/or further designated locations, in particular locations on the perimeter.

The points marked on the first two-dimensional image and/or the second two-dimensional image, respectively, may be obtained from calculations based on a marked structure or based on more than one marked structure (of the same or different kind). These calculations may involve the determination of a center or symmetry point of lines or areas, the formation of averages, etc.

In order to improve the precision of the marking step, a magnified view of the image may be displayed.

The present invention includes variants of the method where more than two (or more than three) points are marked for each of the images as well as variants where more than two images obtained at different points in time are marked. In principle, the number of marked points does not need to be identical for all the processed images.

Depending on the examined property, the two (or more) images may have been obtained at an interval of several minutes, hours, days or even weeks or months. Marking of the points as well as obtaining the third and/or sixth data may be effected quasi-simultaneously with the capture of the respective image or at a later stage, prior to or after the capture of further images. Nevertheless, in order to allow for immediate user feedback it is preferred that marking the fourth and fifth point as well as obtaining the sixth data is quasi-simultaneous with capture of the second two-dimensional image. The immediate user feedback allows to capture further image data until a second image is available that meets the criteria for matching with the first image.

The inventive method ensures that two or more images representing the same tissue volume that are used to examine a condition fulfill certain matching criteria. The two or more images may be taken successively in a single measuring session and/or in independent sessions at a time interval of several minutes up to several months. In the first case, the two or more images may represent the situation at different external or internal influences such as different applied external pressure, different phase of blood circulation or breathing cycle, etc. In the second case, the images may be used to monitor the temporal progression of the condition affecting the tissue volume.

In particular, the matching criteria are chosen in such a way that relevant properties and their change from image to image may be reliably obtained from the images and/or from sensor data. This is particularly relevant for images that are captured in independent sessions, involving independent placement of the imaging device and potentially different operators.

The matching criteria or the associated thresholds may be different for the comparison of two images acquired in the same measuring session and for the comparison of two images acquired in individual measuring sessions. As an example, if a relative property (such as a ratio of distance values) is obtained in each of the individual measuring sessions, a close match of the site and imaging conditions is much more critical with respect to the two or more images of an individual session that are used to obtain the relative property compared to the images from different sessions.

The inventive method is particularly useful for the processing of B-mode ultrasound images, but applicable to various other imaging modalities, including X-ray, OCT, and MRI.

In embodiments of the inventive method, at least one of the first, second, fourth and fifth points is automatically marked based on a structure imaged in the respective image. The automatic marking may be based on known digital image processing techniques, including machine-learning based methods. The structure may be easily identifiable landmarks such as an interface between layers of the imaged tissue, blood vessels, bones, etc.

All of the points mentioned may be automatically marked, especially if they are all related to structures that may be automatically identified within the image data. In a variant, only some of the points are automatically marked, where e.g. the second point is automatically marked based on the manual marking of the first point, and the fourth point is automatically marked based on the manual marking of the third point. After automatic marking of one or several points, a confirmation of the operator may be requested. The operator may be given the opportunity to either confirm the automatic marking or to manually alter the markings or some of them.

In a preferred embodiment of the method, the third data is obtained from marking a third point in the first two-dimensional image, and the sixth data is obtained from marking a sixth point in the second two-dimensional image.

Again, the third and sixth point may be marked manually, wherein the same options are available as described above in connection with the first, second, fourth and fifth point. The third and sixth point may be obtained in a single marking step together with the first and/or second or the fourth and/or fifth point, respectively, e.g. from the marking of an extended 1- or 2-dimensional structure.

As well, the third and sixth point may be automatically marked using the techniques mentioned above in connection with the first, second, fourth and fifth point.

In a preferred embodiment, the third and sixth point are automatically marked whereas the first, second, fourth and fifth point are manually marked. In this case, the marked first and second points may be taken into account for automatically defining and/or marking the third point, whereas the marked fourth and fifth points may be taken into account for automatically defining and/or marking the sixth point. In this case, it is not mandatory to display the third point or sixth point on the respective image, but it may be used for internal purposes only. Nevertheless, in many cases it will be useful to display the points that have been marked by a fully automatic step as well, in order to allow the user to monitor the process.

Nevertheless, also in this case, the manual marking step of the first, second, fourth and fifth point may be assisted based on image processing or segmentation. This may e.g. include the step of providing suggestions for the locations of the first, second, fourth and fifth point, to be confirmed (or possibly altered) by the operator.

Several criteria may be employed to check whether a position and orientation of the first two-dimensional image and the second two-dimensional image sufficiently match. One criterion relates to the intensity or grayscale value, i. e. for the checking step an intensity or grayscale value in a region of the third marked point is compared to an intensity or grayscale value in a region of the sixth marked point. If the difference exceeds a certain threshold, the images will be deemed to be non-matching. The region may be as small as a single pixel at the location of the marked point or—preferably—comprise a certain area surrounding the marked point. The intensities or grayscale values of the points in the area may be suitably averaged, e. g. using a weighted average, where the weight of the pixels decreases with increasing distance from the respective marked point.

Another criterion is based on data of a positional sensor, in particular of a positional sensor integrated to the imaging device, in particular of a hand-held imaging probe. In this case, the third data is obtained from the positional sensor measuring a first position of the image sensor when capturing the first two-dimensional image and in the sixth data is obtained from the positional sensor measuring a second position of the image sensor, when capturing the second two-dimensional image. It is important to note that the second image may be obtained using the same imaging device that has been used to capture the first image or from a different imaging device (and therefore, the sixth data will be obtained by a different positional sensor).

In particular, the first position is a first inclination and the second position is a second inclination, wherein inclinations are preferably two-dimensional or three-dimensional inclinations, representable by two or three Euler angles, respectively.

Instead of the inclinations or in addition to them, the first position may be a first location relative to the tissue, in particular relative to the patient's body surface. Preferably, the location is measured along two directions, e. g. in an XY-cartesian coordinate system.

In a preferred embodiment, the location of a hand-held device being guided over the body surface of the patient, i. e. an ultrasonic probe, is tracked using an optical sensor. This sensor may include a light source, e. g. an infrared laser diode, and an image sensor, e. g. a CCD sensor, similar to an optical computer mouse device. Such a positional sensor is compact, reliable and yields precise results, in particular relating to relative movements of the hand-held device relative to the body surface as long as there is a contact between the device (and thus the sensor) with the body surface. Compared to image-data based tracking, the computational load is drastically reduced. In addition, even if the image data represents a single slice along the extension of an imaging array, the optical sensor allows for tracking the position not only along this extension but also in a direction parallel thereto.

Based on the positional data it may be avoided to process (and compare) views that relate to substantially different imaging planes.

In other variants of the inventive method, the positional data, in particular the location data, may be used to generated three-dimensional data. This data may include volumetric data imaging the tissue in three dimensions. Alternatively or in addition, the extension of a tissue structure (e. g. a compartment) may be tracked along a path travelled by the imaging probe. Based on this detection e. g. a maximum extension along a predetermined direction may be automatically determined.

Within a single measuring session, the sensor may be used to track the movements of the imaging device in order to detect movements that are associated with an impairment of the image quality and/or the match of images taken successively within the session. As an example, if the measuring session foresees the capture of two or more images at different values of external pressure exerted by the imaging device to the examined body portion, a movement along the longitudinal axis of the device is required and acceptable whereas other movements such as slipping movements shifting the measurement site, tilting movements changing the measurement axis and rotational movements changing the imaged plane should be minimized. Accordingly, upon detecting non-acceptable movements from the positional data, a warning may be issued to the operator. The warning may be accompanied by an indication supporting the operator to correct the position and/or orientation of the imaging device, e.g. an arrow indicating the direction (and potentially the extent) of a correction, in particular with the goal of minimizing errors due to a suboptimal positioning during the gradual increase of the external pressure. The warning and/or indication may be provided by a visual, acoustic and/or haptic information directly on the probe and/or the user interface that is used for displaying and marking the images. In the latter case, the information may be shown within and/or aside the real-time image. Generally, visual indications may be light signals, changing colours, graphical symbols (such as arrows or lines), numeric values or similar.

Positional data recorded throughout a single or several measuring sessions may be used to provide a (summary) report to the operator, in particular for improving the precision of the operator's handling in future measuring sessions, wherein the precision may be in particular related to the positioning of the probe and/or the movement for increasing the external pressure.

Alternatively or in addition, a relative twisting of the imaging planes may be detected based on the image data itself, e.g. using speckle tracking techniques. If a vector representing the twist may be obtained from the positional data and/or the image data it may be possible to correct one or both images in order to render a meaningful comparison feasible. In these cases, the two images will be classified as non-matching only if a correction is not possible or if the correction would lead to an intolerable increase of the error margin.

Another criterion relates to the geometric relationships between the marked points and the third and sixth data, respectively. In this case, for the checking step at least one first reference angle is determined from the first and second marked points and the third data and at least one second reference angle is determined from the fourth and fifth marked points and the sixth data, whereupon the first reference angle and the second reference angle are compared. Again, the reference angles are indicative of an orientation of the imaging planes. As well, a substantial mismatch of these angles might indicate other problems with respect to the comparability of the two images.

Another geometric criterion relates to lengths. In this case, for the checking step at least a first reference length is determined from the first and second marked points and the third data and at least a second reference length is determined from the fourth and fifth marked points and the sixth data, whereupon the first reference length and the second reference length are compared. If the location of the marked points, the nature of the third and sixth data and the reference lengths are suitably chosen, these reference lengths will relate to geometric relationships that are basically constant in a given volume of tissue. In this case, if the reference lengths obtained from the first and from the second image differ significantly, this is indicative of a mismatch between the images.

In some embodiments, the first length denotes a distance between the first point and the second point, the second length denotes a distance between the fourth and the fifth point, the first length is normalised based on the third data, and the fourth length is normalised based on the sixth data.

Thereby, a consistent imaging ratio may be ensured, which is relevant especially if absolute values are determined from the two images. The normalization may be based on lengths obtained from the two points and the further data (e.g. a further marked point) and/or on angles obtained from the points and further data that may be indicative of a twist between the imaging planes, as discussed above.

Preferably, in a capturing process of the second two-dimensional image, the first two-dimensional image is displayed to the operator together with a candidate for the second two-dimensional image for supporting the capture of the second two-dimensional image. In particular, the candidate for the second two-dimensional image represents the most recently captured image data (“live view”). This allows for immediate side-to-side comparison of the second image with the first image, thus facilitating the capture of matching images.

It is particularly preferred to display the first and second marked points and a geometric object representing the third data are together with the first two-dimensional image. In particular, if the third data is a point, the geometric object may be a direct representation of this point. If the fourth and fifth point (and potentially the sixth point) are manually marked in the second image, the display of the markings in the first picture will be helpful for the operator and reduce the risk of false markings.

In a preferred embodiment, the fourth and fifth point and a geometric object representing the sixth data are marked in the candidate for the second two-dimensional image, and a result and/or an intermediate result of the verification based on the marked first, second, fourth and fifth point and the third and sixth data is displayed together with the first two-dimensional image. If the sixth data is a point, the geometric object may be a direct representation of this point.

This allows for an iterative finding process when capturing the second picture, e.g. for the correct positioning of an imaging probe. The positioning may be supported by displaying appropriate information, based on the points and/or further data and the intermediate result of the verification. The appropriate information may be visualized by arrows, gauges or other elements.

In some embodiments, in addition to the first, second, fourth and fifth marked point as well as the third and sixth data, seventh data is obtained from a positional sensor measuring a first position of an image sensor when capturing the first two-dimensional image, and eighth data is obtained from a positional sensor measuring a second position of an image sensor, when capturing the second two-dimensional image, wherein the seventh data and the eighth data are used for checking whether the position and orientation of the first two-dimensional image and the second two-dimensional image sufficiently match and/or for determining the first length and/or for determining the second length.

In particular, the first position is a first inclination and the second position is a second inclination, wherein inclinations are preferably two-dimensional or three-dimensional inclinations, representable by two or three Euler angles, respectively.

Instead of the inclinations or in addition to them, the first position may be a first location relative to the tissue, in particular relative to the patient's body surface. Preferably, the location is measured along two directions, e. g. in an XY-cartesian coordinate system.

In a preferred embodiment, the location of a hand-held device being guided over the body surface of the patient, i. e. an ultrasonic probe, is tracked using an optical sensor. This sensor may include a light source, e. g. an infrared laser diode, and an image sensor, e. g. a CCD sensor, similar to an optical computer mouse device. Such a positional sensor is compact, reliable and yields precise results, in particular relating to relative movements of the hand-held device relative to the body surface as long as there is a contact between the device (and thus the sensor) with the body surface. Compared to image-data based tracking, the computational load is drastically reduced. In addition, even if the image data represents a single slice along the extension of an imaging array, the optical sensor allows for tracking the position not only along this extension but also in a direction parallel thereto.

Using additional marked points as well as the data obtained from a positional sensor allows for additional verifications and/or corrections. This embodiment of the inventive method may be combined with any of the optional properties described above, relating to the embodiments including the marking of the third and sixth points in the first and second two-dimensional image, respectively.

Other advantageous embodiments and combinations of features come out from the detailed description below and the entirety of the claims.

### BRIEF DESCRIPTION OF THE DRAWINGS

The drawings used to explain the embodiments show:

FIG. 1 A flowchart of a method of processing two-dimensional images representing a section of a three-dimensional tissue according to the invention;

FIG. 2a, b schematic representations of two embodiments of an imaging device having a positional sensor, suitable for use in the context of the method;

FIG. 3 a schematic representation of a measuring session using the imaging device;

FIG. 4a-c images representing a section of a three-dimensional tissue volume at different external pressure, with marked points relating to a length to be determined and an anatomical landmark;

FIG. 5a-c images representing a section of a three-dimensional tissue volume at different external pressure, with marked points relating to a length to be determined and an anatomical landmark, including reference angles; and

FIG. 6a, b a portion of a graphical user interface of an imaging device, displaying the result of a previous measurement and of a present measurement, including a quality assessment of the present measurement.

In the figures, the same components are given the same reference symbols.

### PREFERRED EMBODIMENTS

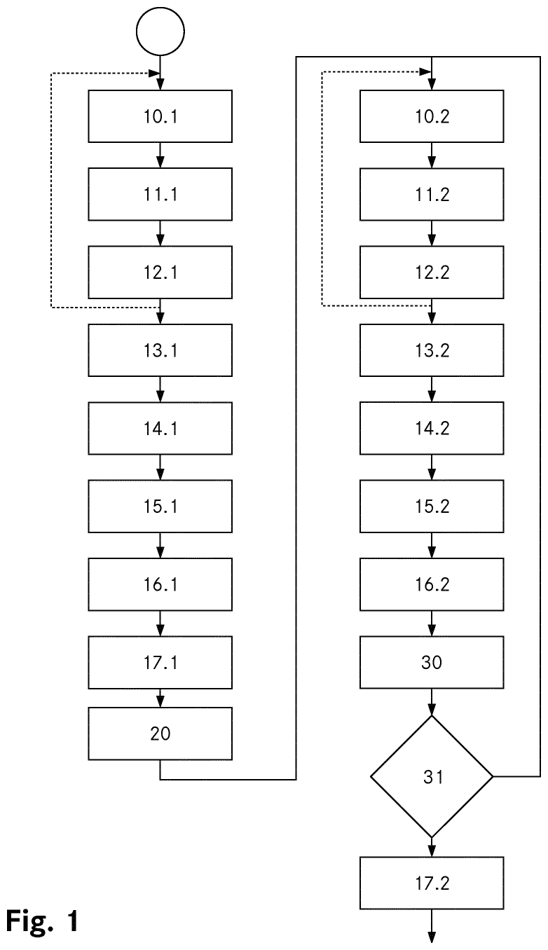

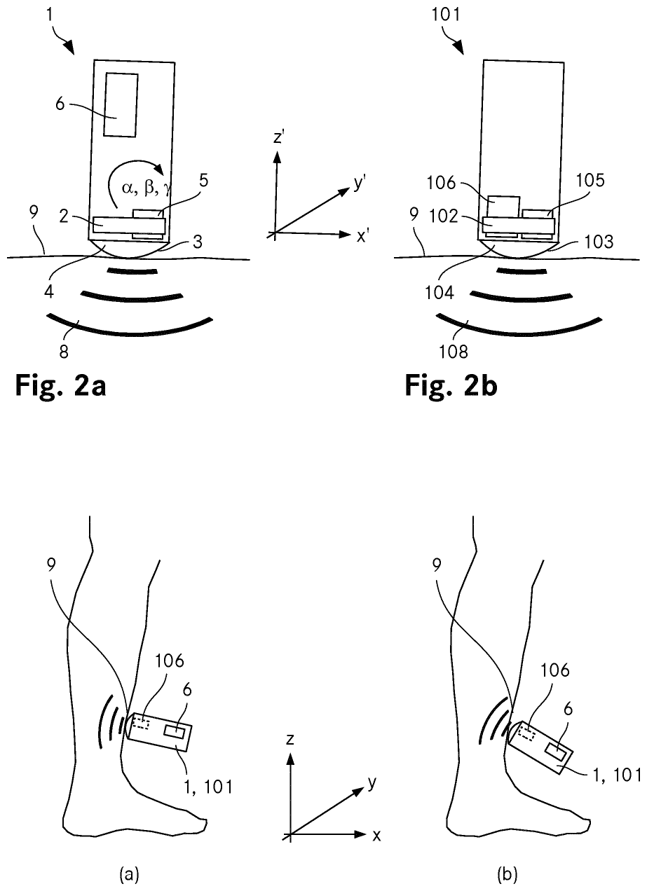

FIG. 1 is a flowchart of a method of processing two-dimensional images representing a section of a three-dimensional tissue according to the invention. The described example relates to a sequence of acquiring two images of the same tissue volume at different external pressure. In the example, the images are obtained from an ultrasound imaging device providing B-mode ultrasound images, schematically represented in FIGS. 2a, 2b. In a first embodiment shown in FIG. 2a, the probe 1 includes an ultrasound array 2 for transmitting and receiving ultrasound waves 8 in a manner known as such. On its contact surface with the skin surface 9 of a subject, the probe 1 is provided with a flexible membrane 3, delimiting a fluid chamber 4. A pressure sensor 5 is provided for measuring the pressure within the fluid chamber 4. A corresponding device is described in co-pending PCT/EP2022/079972 of the same applicant.

Furthermore, the probe 1 is provided with a positional sensor, namely an accelerometer 6. The accelerometer 6 allows in particular for the determination of the orientation of probe 1 in space, about three Euler angles α, β, γ.

In a second embodiment shown in FIG. 2b, the probe 101 includes an ultrasound array 102 for transmitting and receiving ultrasound waves 108 in a manner known as such. On its contact surface with the skin surface 9 of a subject, the probe 101 is provided with a flexible membrane 103, delimiting a fluid chamber 104. A pressure sensor 105 is provided for measuring the pressure within the fluid chamber 104. A corresponding device is described in co-pending PCT/EP2022/079972 of the same applicant.

Furthermore, the probe 101 is provided with a positional sensor, namely an optical sensor 106 including an infrared laser source for illuminating a region of the skin surface 9 below the probe 101 and a CCD sensor for imaging this region. Based on the CCD images, the movement of the probe 101 relative to the skin surface 9, in two directions X, Y, may be identified.

Further embodiments of the probe include an accelerometer as well as an optical sensor, which allows for the determination of both the location as well as orientation of the probe.

Accordingly, a first image is obtained (step 10.1) using probe 1, 101. The first image is pre-processed (step 11.1) on the probe 1 and/or a processing and display device connected to the probe 1, 101. The pre-processed image is then displayed (step 12.1) on a display. Steps 10.1-12.1 are repeated in a circle process to continuously update the displayed image. As soon as the operator chooses to mark a first point (step 13.1), the most recent image is used for the subsequent steps and no new images are acquired and/or displayed.

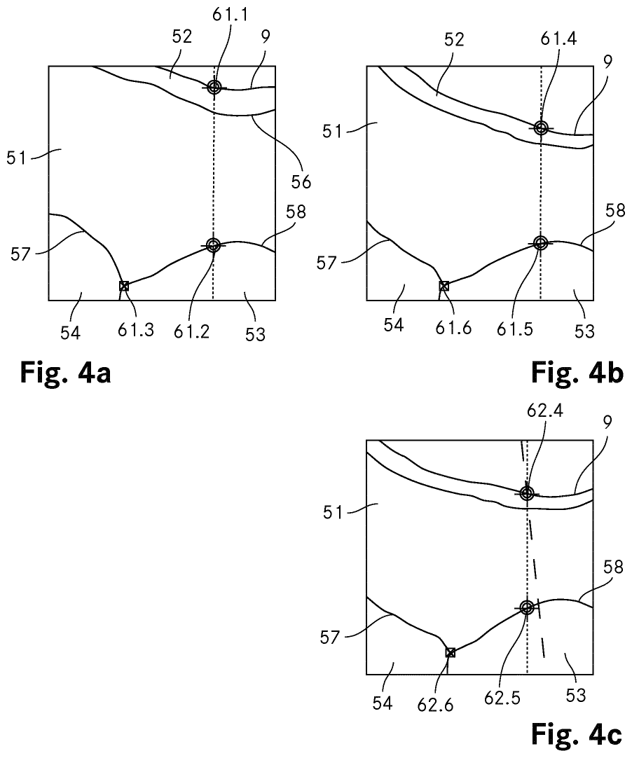

Marking of the first point (step 13.1) is effected manually by the operator moving a cursor on the image, using a suitable input device (e. g. a touch screen, a touchpad, cursor keys etc.) and confirming a certain cursor position by a corresponding action, such as pressing a designated location on a touchscreen, pressing a confirmation button or similar. The confirmed first marked point is displayed on the image, e.g. using a crosshair (cf. FIG. 4a and the description below).

As soon as the first point has been manually marked by the operator, proposed positions for a second point and for a third point are automatically determined and displayed (steps 14.1, 15.1). The proposed positions are determined by processing the image data representing the first image, based on the location of the first point and anatomical structures identified in the image data. The process is described in more detail below, in connection with FIG. 4.

The proposed positions are displayed on the image, together with the first marked point. The operator is then asked to confirm the proposal or to adapt the placement of the proposed positions (step 16.1). Again, this is done using a suitable input device. As soon as all positions are deemed correct by the operator, the choice is confirmed. Now, the positions of the first, second and third point are saved together with the image data representing the first image and potential further data, such as a time stamp and a pressure reading of the pressure sensor 5 at the acquisition time of the first image.

The operator is now requested to adapt a modality of the image acquisition, in particular to increase an external pressure exerted on the skin surface 9 (step 20).

As soon as the modalities are successfully adapted, a second image is obtained (step 10.2) using probe 1, 101, wherein the process is identical to that of acquiring the first image, i. e. the second image is pre-processed (step 11.2) and displayed (step 12.2) on the display. Steps 10.2-12.2 are repeated in a circle process to continuously update the displayed image.

As soon as the operator chooses to mark a fourth point (step 13.2), the most recent image is used for the subsequent steps and no new images are acquired and/or displayed. Based on the position of the first point in the first image, potentially also based on the positions of the second and third point in the first image, and of the image data representing the second image, a proposed position for a fourth point is determined and displayed on the second image. The operator now has the possibility of confirming the proposed position or to move the fourth point prior to confirmation.

As soon as the position of the fourth point is confirmed, proposed positions for a fifth point and for a sixth point are automatically determined and displayed (steps 14.2, 15.2). The proposed positions are determined by processing the image data representing the second image, based on the location of the fourth point, potentially of the first, second and/or third point, and anatomical structures identified in the image data. The proposed positions are displayed on the image, together with the fourth marked point. The operator is then asked to confirm the proposal or to adapt the placement of the proposed positions for the fifth and sixth point (step 16.2). As soon as all positions are deemed correct by the operator, the choice is confirmed.

Next, reference data is generated from the positions of the first to third points, and comparative data is generated from the positions of the fourth to sixth points (step 30). The reference data is compared to the comparative data (step 31). If the comparison indicates that the first and second images and/or the markings in the first and second image sufficiently match, the positions of the fourth, fifth and sixth point are saved together with the image data representing the second image and potential further data, such as a time stamp and a pressure reading of the pressure sensor 5 at the acquisition time of the second image. If the comparison indicates that the match of the first and second image is insufficient, the acquisition and marking of the second image is repeated (step 10.2 et seq.).

As soon as the data relating to two matching images is available, lengths are determined from the positions of the first and second marked point and of the fourth and fifth marked point, respectively. This is described in more detail below, in connection with FIG. 4. The determined lengths are saved together with the other data relating to the first and second image.

The FIG. 3 is a schematic representation of a measuring session using the imaging device. FIG. 3(a) represents the positioning of the probe 1, 101 with respect to the skin surface 9 of a subject, at the time of acquiring a first image. With the probe 1 of the first embodiment, the orientation in space is determined by the accelerometer 6 and stored together with the image data representing the first image (and further data as described above). When acquiring the second image with the same sequence of measurements, as shown in FIG. 3(b), the orientation of probe 1 is continuously tracked by accelerometer 6. As long as the difference of the present orientation from the stored orientation exceeds a certain threshold, a warning is displayed on the display and marking of the fourth and subsequent points will not be possible until a sufficiently matching orientation of probe 1 is achieved. In addition to the warning, further information supporting the operator to find the previous orientation may be displayed (such as arrows indicating the required change of orientation in a certain direction).

With the probe 101 of the second embodiment, the location of the probe 101 relative to the skin surface 9 is continuously determined by the optical sensor 106 and tracked during the sequence of measurements. If the dislocation of the probe 101 during the sequence exceeds a certain limit, a warning will be displayed, and taking further measurements will only be allowed when the the probe 101 has assumed its previous location it had during the first measurement (or a number of previous measurements). In addition to the warning, further information supporting the operator to find the previous location may be displayed (such as arrows indicating the required change of location in a certain direction).

Further checks may be made during a measuring session, based on the positional data. As an example, accelerations and/or velocities of the probe 1 during the session may be monitored and a warning may be issued if they exceed certain limits.

FIGS. 4a-c are images representing a section of a three-dimensional tissue volume at different external pressure, with marked points relating to a length to be determined and an anatomical landmark.

FIG. 4a represents a first image taken at a certain first point in time. In the described example, FIG. 4 represents tissue including the tibial anterior compartment 51, covered by the superficial muscle fascia 52. Behind the tibial anterior compartment 51, the tibia 53 and the interosseous membrana 54 are visible. For simplicity, only the skin surface 9, the interfaces between the mentioned structures, namely the interface 56 between the superficial muscle fascia 52 and the tibial anterior compartment 51 and the interface 57 between the tibial anterior compartment 51 and the interosseous membrana 54, and the edge 58 of the tibia 53, are shown in FIGS. 4-6. In reality, the pre-processed B-mode ultrasonic image will be displayed, and the interfaces or edges may be identified by image processing, depending on the brightness values of the pixels, if needed.

Three points are marked in the first image shown in FIG. 4a, including a first point 61.1 on the skin surface 9, the marking being displayed with a cross-hair shape. A second point 61.2 is marked where an axis running through the first point 61. 1 meets the edge 58 of the tibia 53, the marking of the second point 61.2 again being displayed with a cross-hair shape. A third point 61.3 is marked where the edge 58 of the tibia 53 meets the interface 57 between the tibial anterior compartment 51 and the interosseus membrana 54. This is an anatomical landmark, which allows for easy and reproducible marking. The marking of the third point 61.3 is displayed with a box shape. The markings have been obtained and saved together with the image data by the procedure described above, in connection with FIG. 1.

FIG. 4b represents a second image taken at a certain second point in time. The represented volume of tissue substantially corresponds to the volume represented by the first image, however the external pressure applied to the skin surface 9 of the subject has been increased and thus the compressible structures in the volume, i. e. the tibial anterior compartment 51, the superficial muscle fascia 52 and the interosseus membrana 54, are compressed. Again, three points are marked. These include the fourth point 61.4 on the skin surface 9, the fifth point 61.5 where an axis running through the fourth point 61.4 meets the edge 58 of the tibia 53, and the sixth point 61.6 representing the position where the edge 58 of the tibia 53 meets the interface 57 between the tibial anterior compartment 51 and the interosseus membrana 54. Again, these markings have been obtained and saved together with the image data by the procedure described above, in connection with FIG. 1.

FIG. 4c represents another second image taken at a certain second point in time, with increased external pressure on the skin surface 9. Compared to the image shown in FIG. 4b, the orientation, scale and cutout are different. Again, three points are marked. These include the fourth point 62.4 on the skin surface 9, the fifth point 62.5 where an axis running through the fourth point 62.4 meets the edge 58 of the tibia 53, and the sixth point 62.6 representing the position where the edge 58 of the tibia 53 meets the interface 57 between the tibial anterior compartment 51 and the interosseus membrana 54. Again, these markings have been obtained and saved together with the image data by the procedure described above, in connection with FIG. 1.

In FIG. 4c, the axis between the fourth point 61.4 and the fifth point 61.5 as defined in FIG. 4b is indicated by a dashed line. It is apparent that it runs in a different direction compared to FIG. 4b as well as compared to the markings in the first image shown in FIG. 4a. Apparently, if length between the fourth point 61.4 and the fifth point 61.5 shall be determined, e. g. for comparing this length to a length between the first point 61.1 and the second point 61.2 in the first image, the measurement taken from the positions marked in FIG. 4c will lead to inferior results compared to a measurement taken from the positions marked in FIG. 4b. Accordingly, the markings in FIG. 4c should be declared as invalid and another marking and/or acquisition cycle should be initiated.

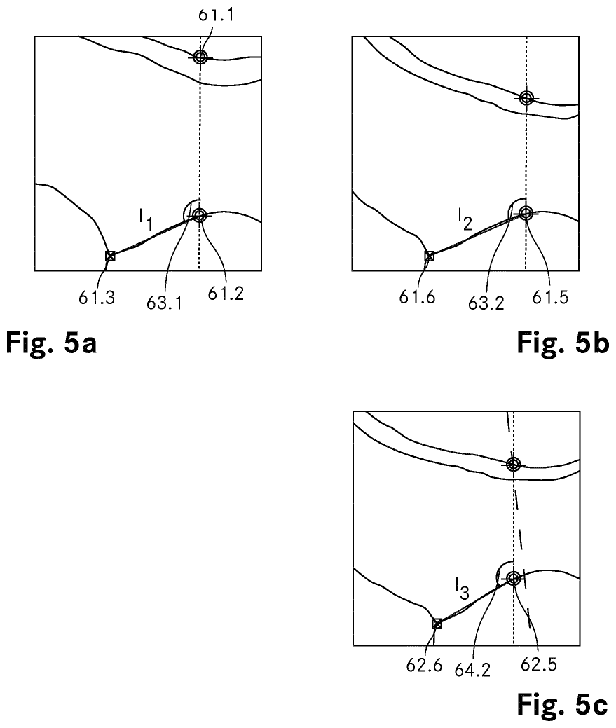

A possible way of assessing the quality of the second image is described in connection with FIGS. 5a-c. These figures basically correspond to FIGS. 4a-c, however, reference angles are added. The reference angle 63.1 in the first image shown in FIG. 5a is an angle between the axis running through the first point 61. 1 and the second point 61.2 and a line connecting the second point 61.2 to the third point 61.3. The value of the reference angle 63.1 is determined as 114.8°. In parallel, the distance I₁ between the second point 61.2 and the third point 61.3, in units relating to the image dimensions, is determined to 43.3% of the image width.

In the second image represented by FIG. 5b, the corresponding comparative angle 63.2 is determined as 114.0° and the distance I₂ between the fifth point 61.5 and the sixth point 61.6 is determined to 46.5% of image width.

In the second image represented by FIG. 5c, the corresponding comparative angle 64.2 is determined as 120.3° and the distance I₃ between the fifth point 62.5 and the sixth point 62.6 is determined to 39.6% of image width.

When comparing the second image as represented by FIG. 5b, or FIG. 5c, respectively to the first image, in a first step the comparative angle is compared to the reference angle. A substantial deviation between the angles indicates that the lengths that shall be determined do not relate to the same axis and/or to the same location in the tissue. If the difference exceeds a certain threshold, e.g. 1.5%, the quality of the second image is deemed to be insufficient and the acquisition is repeated.

In a second step, if the comparison of the angles gave a positive result, the comparison between the comparative length and the reference length may be used to normalize the length measurement in the second image. In the present example, the comparative length is 7.4% bigger than the reference length. Accordingly, assuming isotropic scaling of the images, the measurement of the distance between the fourth point 61.4 and the fifth point 61.5 will be scaled by a factor of 0.931 to compensate for the scaling difference. There may be a further threshold with respect to the comparison between the reference length and the comparative length in order to discard images where the scaling difference is beyond a certain limit. In this case, even if the comparison between the angles is passed the acquisition might have to be repeated due to an excessive difference in scale.

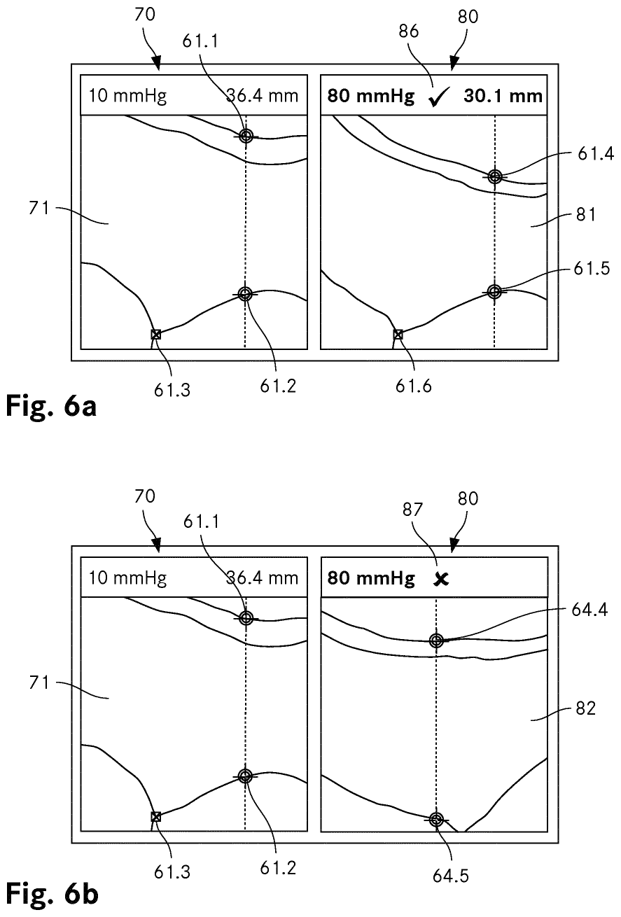

FIGS. 6a, 6b show a portion of a graphical user interface of an imaging device, displaying the result of a previous measurement and of a present measurement, including a quality assessment of the present measurement.

The graphical user interface includes a first display area 70 displaying a first B-mode ultrasound image taken at a first point in time, representing a tissue volume at a first value of external pressure, which is 10 mmHg in the shown example. The graphical user interface further includes a second display area 80 displaying a second ultrasound image taken at a second point in time, after the first point in time, representing essentially the same tissue volume at a second value of external pressure, which is 80 mmHg in the shown example. In both images, points 61.1 . . . 5 have been manually and/or automatically marked, as described above, in connection with FIG. 4.

FIG. 6a shows an example where the second image 81 meets predetermined matching criteria with the first image 71. Accordingly, the second image 81 is accepted, which is indicated by a checkmark symbol 86 above the second display area 80, and the distance between the fourth point 61.4 and the fifth point 61.5 is determined and displayed.

FIG. 6b shows an example where the second image 82 does not meet predetermined matching criteria with the first image 71. Obviously, the axis with the marked points 64.4, 64.5 is on different location than the axis connecting the marked points 61.1, 61.2 in the first image. In the shown case, it was not even possible to mark the sixth point, as the corresponding anatomical landmark was not within the capture image. Therefore, the second image 82 is rejected, which is indicated by a cross symbol 87 above the second display area 80. The acquisition of the second image will thus be repeated.

The invention is not restricted to the embodiments described above. In particular, the validity of the second image may be assessed based on a combination of several criteria, including in particular the results of comparisons between reference and comparative geometric properties (such as angles, lengths or length ratios) as well as measurements taken by sensors such as positional sensors and further properties obtained from the processing of the image data.

The marking process may be different than the one described above. In some embodiments, all points are manually marked whereas in other embodiments the marking process may be completely automatic, without requiring a confirmation by the user.

In summary, it is to be noted that the invention creates a method that allows for a reliable comparison of length values obtained from two or more two-dimensional images representing a section of a three-dimensional tissue volume.

1. A method of obtaining a first length from a first two-dimensional image representing a section of a three-dimensional tissue volume at a first time, and a second length from a second two-dimensional image representing a section of a three-dimensional tissue volume at a second time, comprising the steps of:
marking a first point in the first two-dimensional image;
marking a second point in the first two-dimensional image;
obtaining third data relating to the first two-dimensional image;
marking a fourth point in the second two-dimensional image;
marking a fifth point in the second two-dimensional image;
obtaining sixth data relating to the second two-dimensional image;
checking, using the marked first, second, fourth and fifth points and the third and sixth data, whether a position and orientation of the first two-dimensional image and the second two-dimensional image sufficiently match; and
if yes
determining the first length from the first and second marked point and the third data; and
determining the second length from the fourth and fifth marked point and the sixth data.

2. The method according to claim 1, wherein at least one of the first, second, fourth and fifth points is automatically marked based on a structure imaged in the respective image.

3. The method according to claim 1, wherein the third data is obtained from marking a third point in the first two-dimensional image and that the sixth data is obtained from marking a sixth point in the second two-dimensional image.

4. The method according to claim 3, wherein at least one of the third and sixth point is automatically marked based on the respective image based on an imaged structure.

5. The method according to claim 4, wherein the third and sixth point are automatically marked whereas the first, second, fourth and fifth point are manually marked.

6. The method according to claim 3, wherein for the checking step an intensity or grayscale value in a region of the third marked point is compared to an intensity or grayscale value in a region of the sixth marked point.

7. The method according to claim 1, wherein the third data is obtained from a positional sensor measuring a first position of an image sensor when capturing the first two-dimensional image and the sixth data is obtained from a positional sensor measuring a second position of an image sensor, when capturing the second two-dimensional image.

8. The method according to claim 1, wherein for the checking step at least one first reference angle is determined from the first and second marked points and the third data and at least one second reference angle is determined from the fourth and fifth marked points and the sixth data, whereupon the first reference angle and the second reference angle are compared.

9. The method according to claim 1, wherein for the checking step at least a first reference length is determined from the first and second marked points and the third data and at least a second reference length is determined from the fourth and fifth marked points and the sixth data, whereupon the first reference length and the second reference length are compared.

10. The method according to claim 1, wherein the first length denotes a distance between the first point and the second point, the second length denotes a distance between the fourth and the fifth point, the first length is normalised based on the third data, and the fourth length is normalised based on the sixth data.

11. The method according to claim 1, wherein in a capturing process of the second two-dimensional image, the first two-dimensional image is displayed to the operator together with a candidate for the second two-dimensional image for supporting the capture of the second two-dimensional image.

12. The method according to claim 11, wherein the first and second marked points and a geometric object representing the third data are displayed together with the first two-dimensional image.

13. The method according to claim 11, wherein the fourth and fifth point and a geometric object representing the sixth data are marked in the candidate for the second two-dimensional image and that a result and/or an intermediate result of the verification based on the marked first, second, fourth and fifth point and the third and sixth data is displayed together with the first two-dimensional image.

14. The method according to claim 3, wherein seventh data is obtained from a positional sensor measuring a first position of an image sensor when capturing the first two-dimensional image and in that eighth data is obtained from a positional sensor measuring a second position when capturing the second two-dimensional image, wherein the seventh data and the eighth data are used for checking whether the position and orientation of the first two-dimensional image and the second two-dimensional image sufficiently match and/or for determining the first length and/or for determining the second length.

15. The method according to claim 7, wherein the first position measured by the positional sensor is a first inclination and/or location and the second position is a second inclination and/or location.

16. The method according to claim 15, wherein the positional sensor is integrated into an imaging device.

17. The method according to claim 17, wherein the positional sensor is an optical sensor and a location of the imaging device being guided over a body surface of a patient is tracked using the optical sensor.
